(19) United States
(12) Patent Application Publication
(10) Pub. No.: US 2026/0263088 A1
(43) Pub. Date: Sep. 10, 2026

(54) IMPLANTATION INSTRUMENTS FOR SHOULDER PROSTHESIS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Kenneth Konya, Taylor, TX (US); Michael Seikel, Austin, TX (US); Forrest A. James, Memphis, TN (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/469,596

(22) PCT Filed: Jun. 6, 2024
(86) PCT No.: PCT/US2024/032717

§ 371 Date: Sep. 26, 2025

Related U.S. Application Data
(60) Provisional application No. 63/472,755, filed on Jun. 13, 2023.
(60) Provisional application No. 63/547,455, filed on Nov. 6, 2023.

(51) Int. Cl. A61B 17/16; A61B 17/17; A61B 17/56; A61F 2/40; A61F 2/46

(52) U.S. Cl. CPC A61B 17/1684; A61B 17/1778; A61F 2/4612; A61B 2017/564; A61F 2002/4022; A61F 2002/4687

(57) ABSTRACT

A broach tool for use in preparing an implantation site of a humeral head for implantation of a humeral prothesis therein includes a cylindrical sleeve that fits concentrically around a pin anchored in the implantation site, so that the broach is guided by the pin, and a broach head that has an outer profile formed on the portion thereof that faces towards and is driven against the implantation site. This outer profile engages against the implantation site to form alignment features, or slots, in the implantation site. These alignment features aid a surgeon in properly aligning a compactor with the implantation site after the pin has been removed, the compactor being used for forming the interior of the implantation site to allow the insertion of the humeral prosthesis therein.

### CROSS-REFERENCE TO RELATED APPLICATIONS

This application is a non-provisional of, and claims the benefit of the filing date of, U.S. provisional patent application No. 63/472,755, which was filed Jun. 13, 2023, and U.S. provisional patent application No. 63/547,455, which was filed Nov. 6, 2023. Each of these applications is incorporated herein in its entirety.

### FIELD OF THE DISCLOSURE

The present disclosure relates generally to orthopedic tools and methods for preparing bone surfaces for a joint replacement prosthesis. Specifically, the present disclosure relates to systems and methods for forming a reference point on and/or within a bone to allow for compaction of the bone in a direction that is not perpendicular to the plane in which the bone surface has been cut or resected.

### BACKGROUND OF THE DISCLOSURE

During conventional bone preparation procedures for humeral prosthesis implantation, surgeons must work in two directions to prepare the implantation site on the humeral head for the implantation of the prosthesis therein and/or the attachment of the prosthesis thereto. When performing such conventional bone preparation procedures, it is necessary to ensure that, during compaction for preparing an implant cavity, the compactor is maintained in a prescribed orientation (e.g., angle) based on the selected prosthesis being implanted into the humerus and also the anatomy (e.g., shape of the humerus of the patient) of the patient. Failure to maintain the prescribed orientation can cause varus/valgus and/or flexion/extension misalignments that would cause the prosthesis to not fit properly onto/within the implantation site. However, there is no clear landmark for a surgeon to reference to ensure that the compactor is being driven into the implantation site at the appropriate angle. Thus, a need exists for implantation systems and methods that form landmarks or reference points within a surgical implantation site that allow a surgeon to more readily ensure that the compactor is driven into the implantation site in the prescribed orientation.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Disclosed herein is a broaching system configured for use in preparing an implantation site for implantation of a humeral prosthesis therein. The broaching system includes at least a broach and an osteotome. The broach includes a broach head, a broach stem, and an osteotome slot. The osteotome is configured for removable insertion into and through the osteotome slot for forming one or more alignment features within the implantation site to aid a surgeon to properly align a compactor with the implantation site.

In any of the preceding or subsequent examples, the osteotome slot is formed at an inclined, non-zero angle relative to a longitudinal axis of the broach stem.

In any of the preceding or subsequent examples, the osteotome slot is continuous and uninterrupted from the broach stem to and through the broach head.

In any of the preceding or subsequent examples, the osteotome has a length such that, when the osteotome is inserted fully into the osteotome slot, a distal portion of the osteotome protrudes out of the osteotome slot and into the implantation site to form the one or more alignment features.

In any of the preceding or subsequent examples, the broach stem and the broach head each have a bore formed therein that is configured to receive therein a pin that is implanted within the implantation site, the bore being continuous and uninterrupted within and between the broach stem and the broach head.

In any of the preceding or subsequent examples, the osteotome has a U-shaped profile, with two legs that extend substantially parallel to each other and a base that extends between and connects the two legs, such that the two legs are spaced apart from each other by the base.

In any of the preceding or subsequent examples, the osteotome slot is formed around and is not coincident with the bore.

In any of the preceding or subsequent examples, the broach head has a protruding ridge that extends away from the broach stem and is configured for forming a further alignment feature within the implantation site

In any of the preceding or subsequent examples, the protruding ridge has, on one side thereof, a curved edge and, on the opposite side thereof, an inclined edge that forms an obtuse angle relative to the bottom surface of the protruding ridge that connects the curved edge to the inclined edge.

In any of the preceding or subsequent examples, the one or more alignment features extend substantially coaxially with a longitudinal axis of a humerus in which the implantation site is formed.

Also disclosed herein is a method for performing a broaching procedure to prepare an implantation site for implantation of a humeral prosthesis therein. The method includes providing a broach and providing an osteotome. The broach includes a broach head, a broach stem, and an osteotome slot. The method includes inserting the osteotome into and through the osteotome slot for forming one or more alignment features within the implantation site to aid a surgeon to properly align a compactor with the implantation site. The osteotome is inserted within the osteotome slot in a manner than allows subsequent removal of the osteotome from the osteotome slot without damaging the one or more alignment features formed by the osteotome.

In any of the preceding or subsequent examples, the osteotome slot is formed at an inclined, non-zero angle relative to a longitudinal axis of the broach stem.

In any of the preceding or subsequent examples, the osteotome slot is continuous and uninterrupted from the broach stem to and through the broach head.

In any of the preceding or subsequent examples, the osteotome has a length such that, when the osteotome is inserted fully into the osteotome slot, a distal portion of the osteotome protrudes out of the osteotome slot and into the implantation site to form the one or more alignment features.

In any of the preceding or subsequent examples, the broach stem and the broach head each have a bore formed therein; and the bore is continuous and uninterrupted within and between the broach stem and the broach head. The method includes receiving, within the bore, a pin that is implanted within the implantation site to maintain a prescribed alignment of the broach to the implantation site.

In any of the preceding or subsequent examples, the osteotome has a U-shaped profile, with two legs that extend substantially parallel to each other and a base that extends between and connects the two legs, such that the two legs are spaced apart from each other by the base.

In any of the preceding or subsequent examples, the osteotome slot is formed around and is not coincident with the bore.

In any of the preceding or subsequent examples, the broach head has a protruding ridge that extends away from the broach stem for forming a further alignment feature within the implantation site

In any of the preceding or subsequent examples, the protruding ridge has, on one side thereof, a curved edge and, on the opposite side thereof, an inclined edge that forms an obtuse angle relative to the bottom surface of the protruding ridge that connects the curved edge to the inclined edge.

In any of the preceding or subsequent examples, the one or more alignment features extend substantially coaxially with a longitudinal axis of a humerus in which the implantation site is formed.

Examples of the present disclosure provide numerous advantages. In one non-limiting example advantage, a user of the systems disclosed herein can reliably ensure proper alignment of a broach with an implantation site while simultaneously being able to form alignment features within the implantation site by the insertion of an osteotome through the osteotome slot formed in the broach. In another non-limiting example advantage, the osteotome can be removed from the osteotome slot, thereby allowing the formation of the alignment features within the implantation site that are aligned with the longitudinal humeral axis, then allowing for disengagement of the broach from the implantation site after the osteotome is removed from the osteotome slot, so as to not damage the alignment features formed by the osteotome during disengagement of the broach from the implantation site. Thus, it is advantageous to be able to form alignment features within the implantation site that extend in different directions from each other.

Further features and advantages of at least some of the examples of the present invention, as well as the structure and operation of various examples of the present invention, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 shows a portion of a humerus bone with a portion of the humeral head resected therefrom for implantation of a prosthesis at/in the implantation site formed by the resection;

FIG. 2 shows the implantation site formed on the humeral head, with a sizing template installed over the implantation site and a pin installed in the humeral head in a central location as defined by the sizing template;

FIG. 3 shows a reamer installed concentrically over and around the pin;

FIG. 4 shows the reamer of FIG. 3 applied to the implantation site to remove a portion of the interior bone mass of the humeral head at the implantation site;

FIG. 5 shows the implantation site after the reamer has been removed, with the pin not shown to more clearly illustrate the shape of the implantation site after the reaming procedure has been performed;

FIG. 6 shows a broach according to the presently disclosed subject matter installed concentrically over and around the pin;

FIG. 7 shows the broach of FIG. 6 fully engaged within the implantation site during a broaching procedure to form a broached implantation site;

FIG. 8 shows an osteotome that is insertable within the osteotome slot of the broach shown in FIGS. 6 and 7 for forming a lateral alignment slot internal to the humerus at the implantation site;

FIG. 9 shows another example for a broach with an osteotome slot formed therein.

FIG. 10 shows the broached implantation site after the broaching procedure, including after insertion and removal of the osteotome from the osteotome slot of the broach during the broaching procedure;

FIG. 11 shows a first stage compactor for performing an initial compaction step of the interior bone mass of the humeral head to have a prescribed shape at the implantation site;

FIG. 12 shows the first stage compactor of FIG. 11 being aligned with and partially engaged with the alignment features of the broached implantation site;

FIG. 13 is an internal sectional view of the first stage compactor of FIG. 11 fully engaged within the humeral head at the broached implantation site to form an initially compacted implantation site.

FIG. 14 shows a second stage compactor for performing a further compaction step of the interior bone mass of the humeral head to have a prescribed shape at the implantation site, the second stage compactor being shown aligned with the alignment features of the implantation site, which are formed by the first stage compactor, for insertion of the second stage compactor into the implantation site;

FIG. 15 is an internal sectional view of the humeral head, with the second stage compactor inserted to a depth of a base of the initially compacted implantation site;

FIG. 16 is an internal sectional view of the humeral head, with the second stage compactor having been fully driven into the initially compacted implantation site to form a prepared implantation site; and

FIG. 17 shows an example of a prosthesis that can be inserted into the prepared implantation site of FIG. 16 as part of a prosthetic implantation procedure.

FIG. 18 is a side view of a portion of the prosthesis of FIG. 17, with the prosthetic humeral head omitted from the prosthesis.

FIGS. 19A-D show steps performed using another example of a broach to guide insertion of a second guide pin within the humeral shaft, the second guide pin being used to guide a movement of the example compactors disclosed herein to maintain a proper orientation of such compactors with the implantation site.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

The subject matter described herein relates generally to a surgical broach that aids, in conjunction with an osteotome that is removably insertable within an osteotome slot formed in the broach, in ensuring proper alignment of a compactor with the implantation site in a compaction step that is performed after removal of the surgical broach from the implantation site. In a conventional broaching procedure, the broach is driven into the internal bone mass to form an initial vertical alignment slot. However, there is no landmark that indicates to a surgeon at what angle the first stage compactor should be inclined relative to the planar surface of the implantation site to ensure that the first stage compactor is properly aligned with the longitudinal humeral axis. Failure to form the void/cavity at the proper angle within the humeral head and humeral shaft is known to result in an anatomically incorrect alignment of the prosthesis relative to the implantation site and/or humeral head, which can cause reduced and/or painful post-operative mobility of the joint for the patient, requiring further treatment. Thus, the example surgical broach and osteotome disclosed herein are useful as a system to form visual landmarks that aid a surgeon in ensuring proper alignment of a surgical compactor with the humeral shaft to, in turn, ensure proper fixation of the prosthesis to the humeral head.

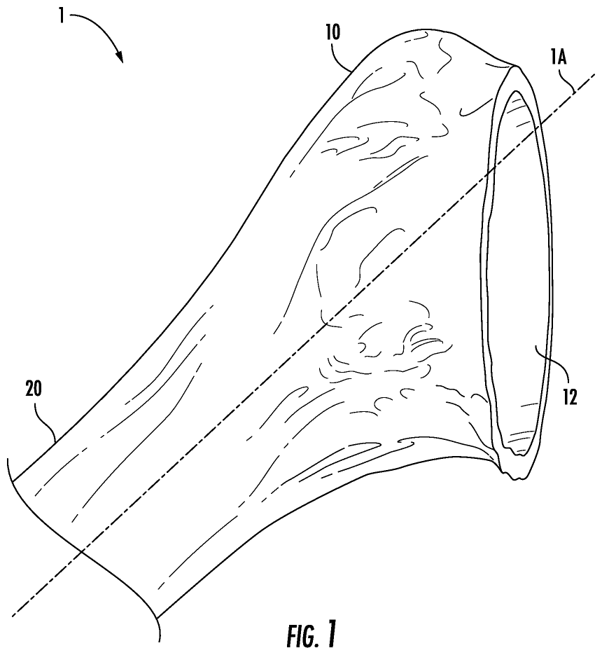

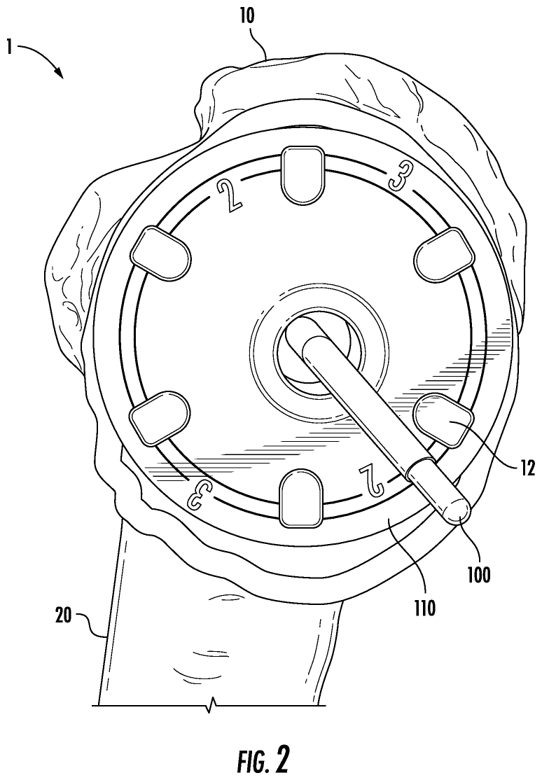

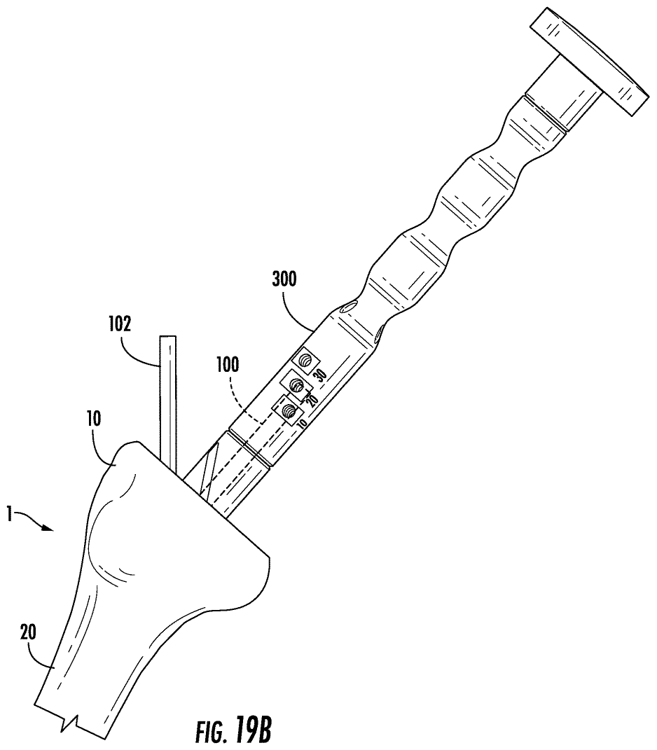

FIG. 1 shows a humerus, generally designated 1, with a humeral head 10 and a humeral shaft 20, the humeral shaft 20 extending in the direction of the longitudinal humeral axis 1A. As a first step, a portion of the humeral head 10 to be replaced with the prosthesis (see, e.g., 700, FIGS. 17 and 18) is first identified and then resected, as shown in FIG. 1, to form a base implantation site 12. The base implantation site 12 has a substantially planar surface across the outer surface (e.g., the surface created during resection of the portion of the humeral head 10) thereof. Next, one or more templates 110 are then, as shown in FIG. 2, applied over the base implantation site 12 to determine a size of the base implantation site 12. The template 110 that is determined to best fit the base implantation site 12, as determined by the surgeon, determines the size of the prosthesis to be implanted in the humerus 1 of the patient. After the appropriate template 110 has been selected, a longitudinally-extending pin 100 is inserted into the base implantation site 12, at a location designated by the template 110 (e.g., the center of the base implantation site 12). Thus, the placement of the pin 100 is generally determined by the selection of the appropriate template 110 by the surgeon. The pin 100 is oriented at a generally 90°angle relative to the plane defined by the outermost surface of the base implantation site 12.

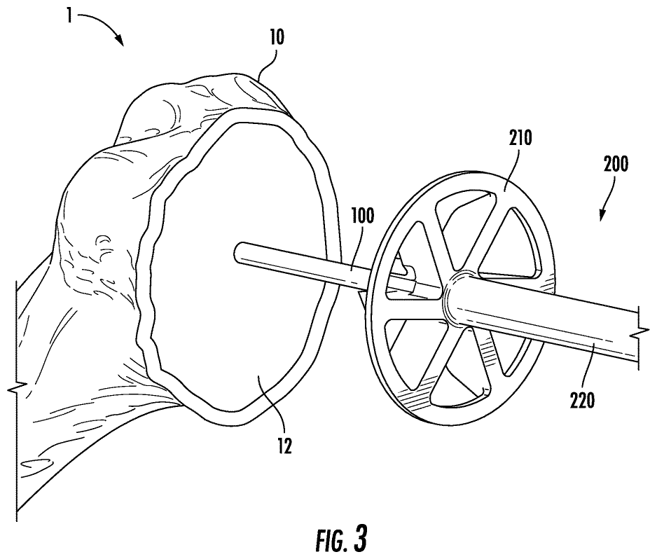

Next, as shown in FIG. 3, a reamer, generally designated 200, is inserted over and around the pin 100 (e.g., coaxially) to maintain a substantially perpendicular alignment of the reamer 200 relative to the base implantation site 12. The reamer 200 has a reamer head 210 that is rigidly attached to a reamer stem 220. Alignment of the reamer with the base implantation site 12 is maintained by the reamer stem 220 being in the form of a hollow cylinder (e.g., having a longitudinally-extending hollow bore formed along the entire length of the reamer stem 220). The bore of the reamer stem 220 advantageously has an internal diameter that is larger, to at least some degree, than the diameter of the pin 100. The bore of the reamer stem 220 extends, in a continuous and uninterrupted manner, along the entire length of the reamer 200, including through the reamer head 210 and the reamer stem 220. The hollow bore of the reamer 200 receives the pin 100 therein. The reamer head 210 has cutting surfaces that, when the reamer head 210 is rotated (e.g., by a rotary tool) against the base implantation site 12, removes internal bone material (e.g., within the base implantation site 12).

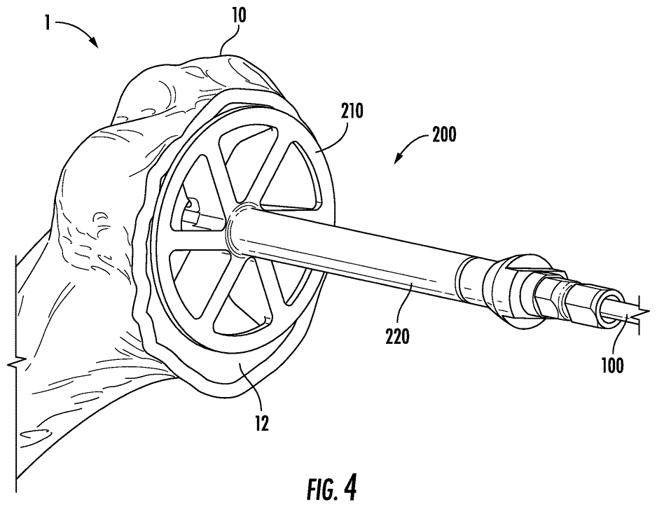

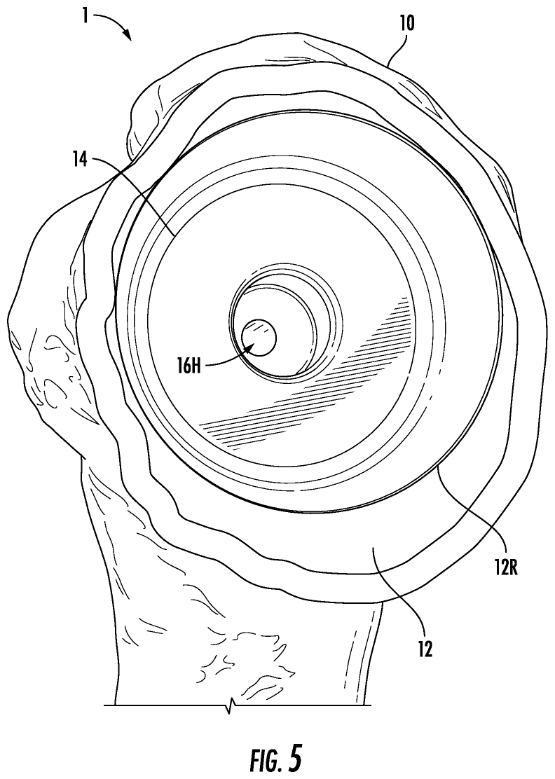

During the reaming procedure, the reamer 200 is advanced along the longitudinal axis of the pin 100, from the position shown in FIG. 3 to the position shown in FIG. 4, in which the reamer head 210 is substantially coplanar with the base implantation site 12 in the plane defined where the reamer head 210 is joined to the reamer stem 220. Thus, when reamer 200 is in the position shown in FIG. 4, the cutting surface(s) of the reamer head 210 have formed a cavity 14, or “bowl region,” which is shown in FIG. 5. The cavity 14 has a prescribed shape and depth that extends from the resection plane that initially defines, as shown in FIG. 1, the base implantation site 12 on the humeral head 10, thus forming the reamed implantation site 12R, as shown in FIG. 5.

The reamer head 210 has a circumferential flange that extends radially about (e.g., away from) the upper edge of the reamer head 210 to define a stop surface, thereby aiding the surgeon in ensuring that the cavity 14 is reamed to the prescribed depth and also aiding the surgeon in ensuring that the reamer is not advanced too far along the longitudinal axis of the pin 100, which would cause the cavity 100 to have too great of a depth. FIG. 5 shows the reamed implantation site 12R, as well as the cavity 14 or bowl region thereof, that is formed by the reamer 200. The reamed implantation site 12R is shown in FIG. 5 with the pin 100 omitted for clarity of illustration, however, the pin 100 is not removed from the humeral head 10 immediately after the reaming procedure is completed.

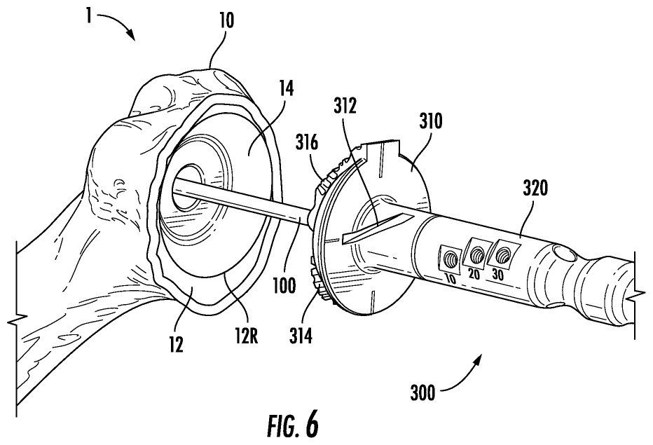

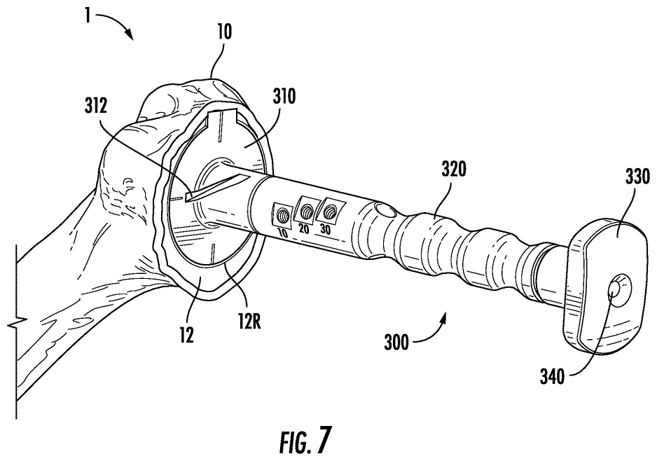

The reamer 200 is then removed from the pin 100 and, as shown in FIG. 6, a broach, generally designated 300, is then inserted over and around the pin 100 (e.g., coaxially) to maintain a substantially perpendicular alignment of the broach 300 relative to the pin 100 and, thus, to the reamed implantation site 12R, at least since the pin 100 is rigidly inserted within the reamed implantation site 12R. The broach 300 has a broach stem 320 that is generally in the form of a hollow cylinder that has an outer wall with a longitudinally-extending bore formed along the entire length of the broach stem 320, so that the pin 100 can pass through the bore 340. The outer wall of the broach stem 320 can, but is not required to, have a generally annular shape or profile. The bore 340 advantageously has an internal diameter that is larger, to at least some degree, than the diameter of the pin 100. The bore 340 extends, in a continuous and uninterrupted manner, along the entire length of the broach 300, including through the broach head 310 and the broach stem 320. The hollow bore 340 of the broach 300 receives the pin 100 therein. Thus, the pin can pass through the portion of the bore 340 within the broach head 310 and into and/or through the portion of the bore 340 within the broach stem 320.

The broach 300 also has, attached to the proximal end of the broach stem 320, a broach head 310. The broach head 310 has a bowl portion 314 that is shaped substantially similarly to the cavity 14 of the reamed implantation site 12R, as shown in FIGS. 5 and 6. The broach head 310 also has a protruding ridge 316 that is formed on and extends away from an outer surface of the bowl portion 314. The protruding ridge 316 is configured to contact the internal surfaces of the reamed implantation site 12R to form a vertical alignment slot, generally designated 16V (see, e.g., FIG. 10) within the reamed implantation site 12R of the humeral head 10 to aid in insertion of the prosthesis (see, e.g., 700, FIG. 17) within a prepared implantation site. The broach stem 320 also has, at the distal end thereof, an impactor surface 330 formed integrally (e.g., such that the entire broach 300 has a unitary structure). The impactor surface 330 is a surface that allows a surgeon to strike the broach 300 to thereby drive the broach head 310 into and against the inner surface of the cavity 14 at the reamed implantation site 12R. The broach stem 320 is advantageously longer than the portion of the pin 100 that protrudes from the surface of the reamed implantation site 12R, so that no portion of the pin 100 protrudes through and/or beyond the impactor surface 330 while the broach 300 is in use (e.g., being struck by a surgeon). After the broach 300 is properly aligned with the reamed implantation site 12R, the impactor surface 330 is impacted until the surface of the broach head 310, by which the broach head 310 is attached to the broach stem 320, is flush with the outer surface of the reamed implantation site 12R.

The broach 300 also has an osteotome slot 312 that extends from the broach stem 320 into the broach head 310. The osteotome slot 312 is inclined at a non-zero angle relative to the longitudinal axis of the broach stem 320. In one example, the non-zero angle can be between about 1° to about 90°. In a more specific example, the non-zero angle can be between about 25° to about 75°. In a still more specific example, the non-zero angle can be between about 30° to about 70°. In a yet more specific example, the non-zero angle can be between about 45° to about 60°. In another example, the non-zero angle can be between about 30° to about 45°. The osteotome slot 312 is formed in and/or through the broach head 310. In the example broach 300 illustrated herein, the osteotome slot 312 is formed partially in and around the outer wall of the broach stem 320. In the example shown herein, no portion of the osteotome slot 312 is coincident with any portion of the bore 340 of the broach 300, either in the broach head 310 or in the broach stem 320. The osteotome slot 312 is shaped to receive an osteotome, generally designated 400, within the osteotome slot 312.

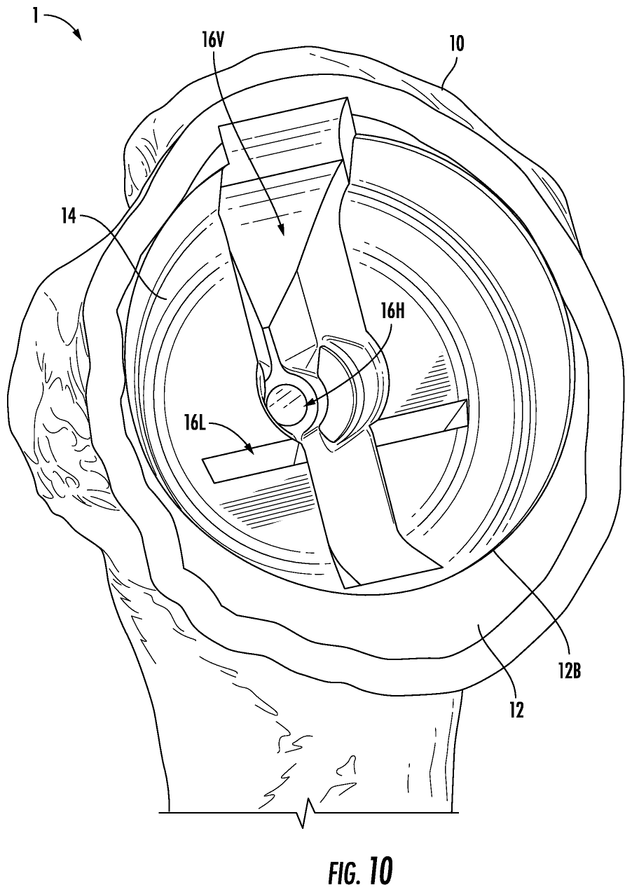

The osteotome 400 has a generally U-shaped profile, as can be seen in FIG. 8. Thus, the osteotome slot 312 is formed as two generally longitudinally-extending portions into and through which the “legs” of the U-shaped base of the osteotome 400 are inserted. These portions of the osteotome slot 312 are formed around and on opposite sides of the bore 340 in the broach stem 320 and broach head 310. Thus, a first portion of the osteotome slot 312 is formed on an opposite side of the bore 340 from the second portion of the osteotome slot 312.

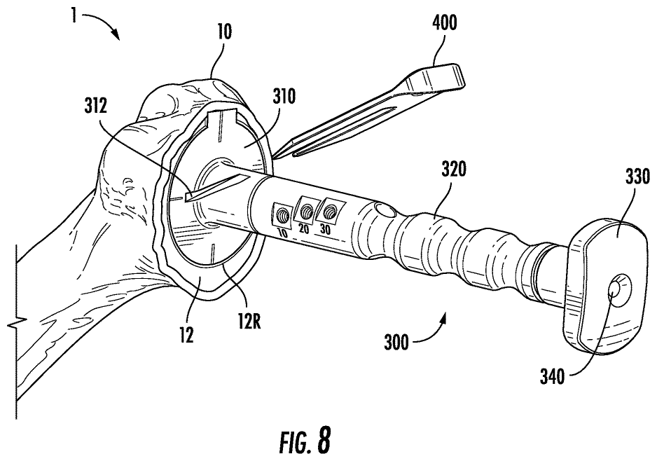

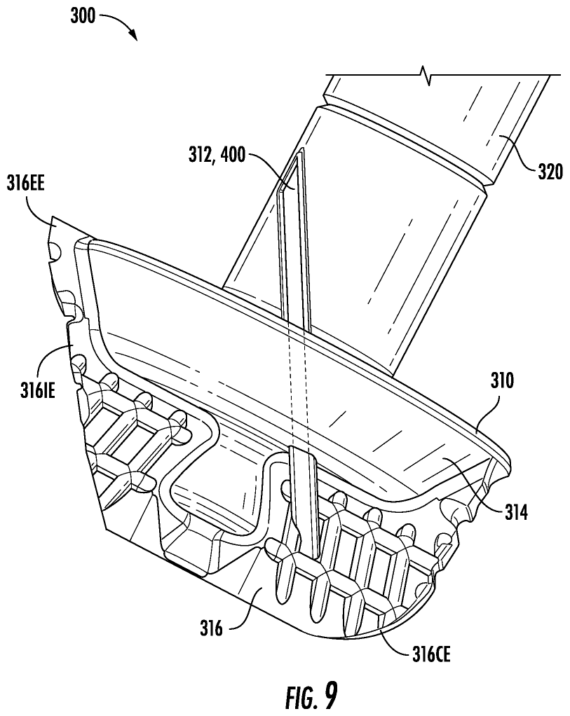

A plurality of broaches 300, each with a different angle of inclination for the osteotome slot 312, can be provided. The broach 300 is selected by the surgeon based on the anatomical shape of the humerus 1, which can be determined during resection of the humeral head 10. The broach 300 that has an angle of inclination of the osteotome slot 312 that is the closest (e.g., out of all of the broaches 300 provided to the surgeon) to parallel, or at least substantially parallel (e.g., ±15°, ±10°, ±5°, ±3°, ±2°, or ±1°), to the longitudinal humeral axis 1A is selected for use during the broaching procedure. Thus, when the broach 300 is driven into the reamed implantation site 12R, as shown in FIG. 7, the osteotome slot 312 of the broach 300 is advantageously parallel to, or at least substantially parallel to, the longitudinal humeral axis 1A.

While the broach 300 is held in the position shown in FIG. 7, the osteotome 400 shown in FIG. 8 is aligned with the osteotome slot 312 and the “legs” of the osteotome 400 are each inserted into a corresponding one of the portions of the osteotome slot 312. The osteotome 400 is fully inserted into the osteotome slot 312, which may require application of force, such as, for example, using a mallet to strike the base of the osteotome 400, such that the base of the U-shaped profile is in contact (e.g., direct contact) with the outer surface of the outer wall of the broach stem 320 when the osteotome 400 is fully inserted within the osteotome slot 312. As used herein, the “base” of the U-shaped profile of the osteotome 400 is the portion that is directly attached to both of the “legs” of the osteotome 400, such that the “legs” extend away from the “base” at the opposing ends of the “base” of the osteotome 400. When fully inserted into the osteotome slot 312, the osteotome 400 forms a lateral alignment slot (see 16L, FIG. 10). The “legs” of the U-shaped profile of the osteotome 400 are advantageously spaced apart from each other by a distance that is the same as or less than a width of the protruding ridge 316, such that the lateral alignment slot(s) 16L intersect(s) with (e.g., forming a continuous cavity with) the vertical alignment slot 16V, as shown in FIG. 10. The lateral alignment slots 16L can, in some instances, be referred to herein as “anterior” and “posterior” slots, respectively.

The osteotome 400 is removed from the osteotome slot 312 before the broach 300 is removed (e.g., by sliding along the pin 100) from the broached implantation site 12B, so as to advantageously avoid the osteotome 400 from disturbing the broached implantation site 12B during removal of the broach 300 from the broached implantation site 12B. After removal of the osteotome 400 from the osteotome slot 312 and, subsequently, of the broach 300 from the broached implantation site 12B, the broach 300 and the osteotome 400 have formed the broached implantation site 12B, an example of which is shown in FIG. 10.

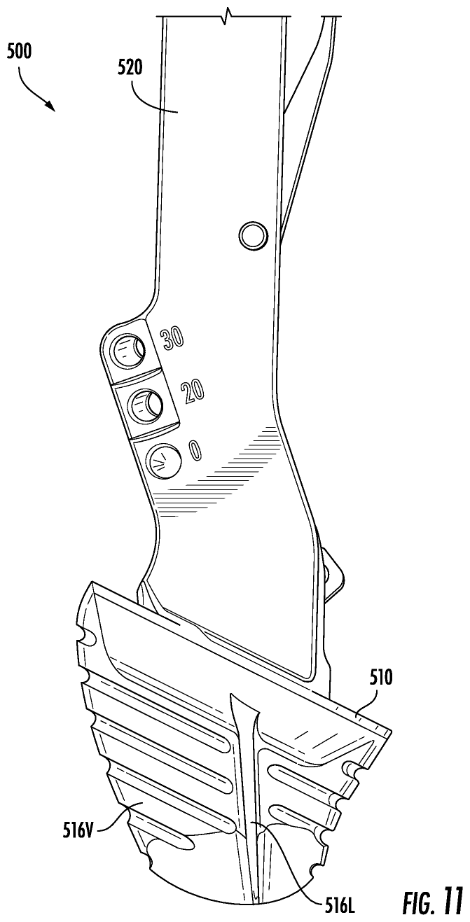

FIG. 9 shows a sectional view of an example broach 300. The broach head 310 has a bowl portion 314 and is attached to the broach stem 320, as is shown in FIGS. 6-8. However, in the broach 300 of FIG. 9, the protruding ridge 316 is larger and differently shaped than for the broach shown in FIGS. 6-8. The protruding ridge 316 has, on one side, a curved edge 316CE and, on the opposite side, an inclined edge 316IE. The broach head 310 is advantageously positioned such that the curved edge 316CE extends towards (e.g., is closest to, relative to the inclined edge) the humeral shaft 320. The inclined edge 316IE defines an extended edge 316EE that extends radially outwardly by a prescribed distance, beyond the outer circumferential edge of the bowl portion 314, so as to form a portion of the vertical alignment slot (see, e.g., 16V, FIG. 10) to have a space or region that allows for insertion of the ridge area (see 718, FIG. 18) of the prosthesis base. The broach stem 320 and broach head 310 shown in FIG. 9 have an osteotome slot 312 formed therethrough, the broach 300 shown in FIG. 9 having an osteotome 400 shown inserted within the osteotome slot 312. The shape and functionality of the osteotome 400 and osteotome slot 312 of the example broach 300 shown in FIG. 9 is substantially identical to the osteotome 400 and osteotome slot 400 of the example broach 300 shown in FIGS. 6-8.

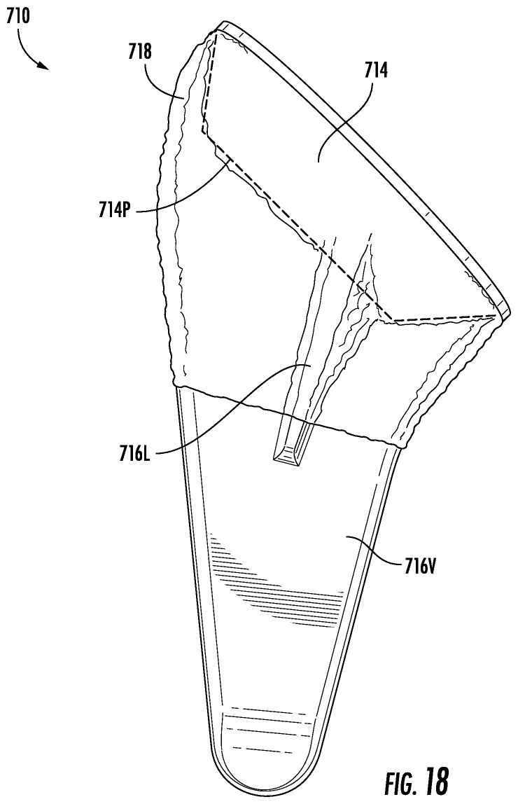

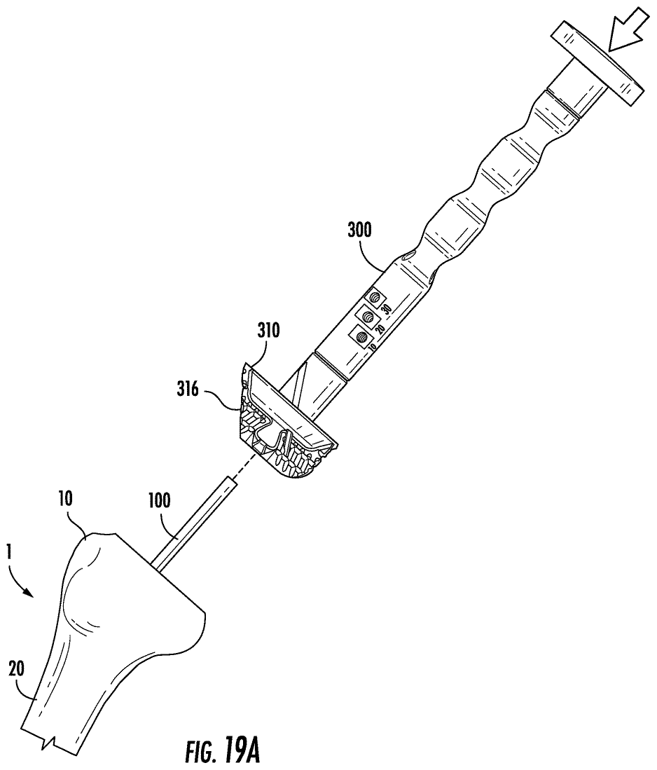

The placement of the broach 300 and the insertion of the osteotome 400 within the osteotome slot 312 determines the final position of the prosthesis (see 700, FIG. 17) being implanted. In particular, for humeral prosthetic implantation procedures, the direction of extension of the humerus (i.e., the longitudinal humeral axis 1A) is inclined at a non-zero and non-perpendicular angle relative to the plane in which the implantation site 12 is formed. In order to ensure adequate fixation of the prosthesis 700 within the humerus 1, it is necessary for the compactor (see 500, FIGS. 11-13, and 600, FIGS. 14-16) to be driven into the broached implantation site 12B at this inclined angle relative to the plane of the outer surface of the broached implantation site 12, such that a cavity with lateral and vertical alignment slots 16L, 16V corresponding to the alignment features of the prosthesis 700 to be inserted therein can be formed internal to the humerus 1 for fixation of the prosthesis 100 implanted therein, by fasteners (e.g., screws) and also ossification. The formation of alignment features (e.g., lateral and vertical alignment slots 16L, 16V) that extend in two different planes within the broached implantation site 12B before compaction advantageously allows a surgeon to ensure that the first stage compactor 500 (see FIGS. 11-13) is also aligned with the longitudinal humeral axis 1A by simultaneously aligning the vertical fins 516V of the first stage compactor 500 with the vertical alignment slot 16V and also the lateral fins 516L of the first stage compactor 500 with the lateral alignment slots 16L, ensuring proper alignment of the first stage compactor 500 relative to the longitudinal humeral axis 1A in at least two (2) dimensions or planes.

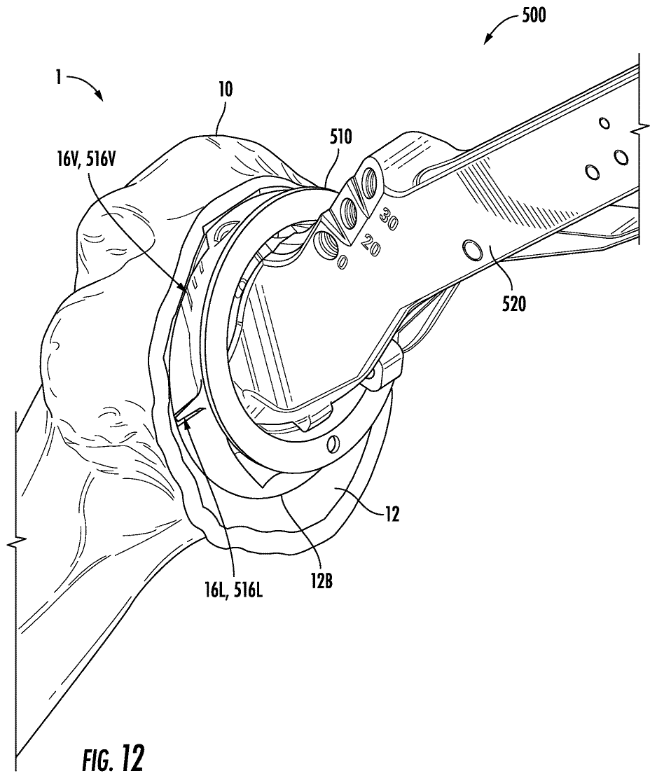

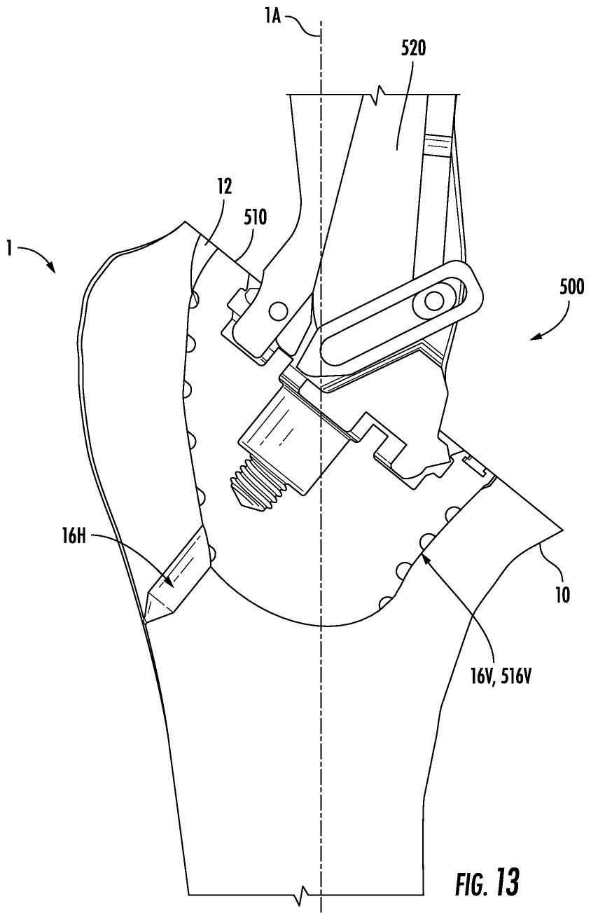

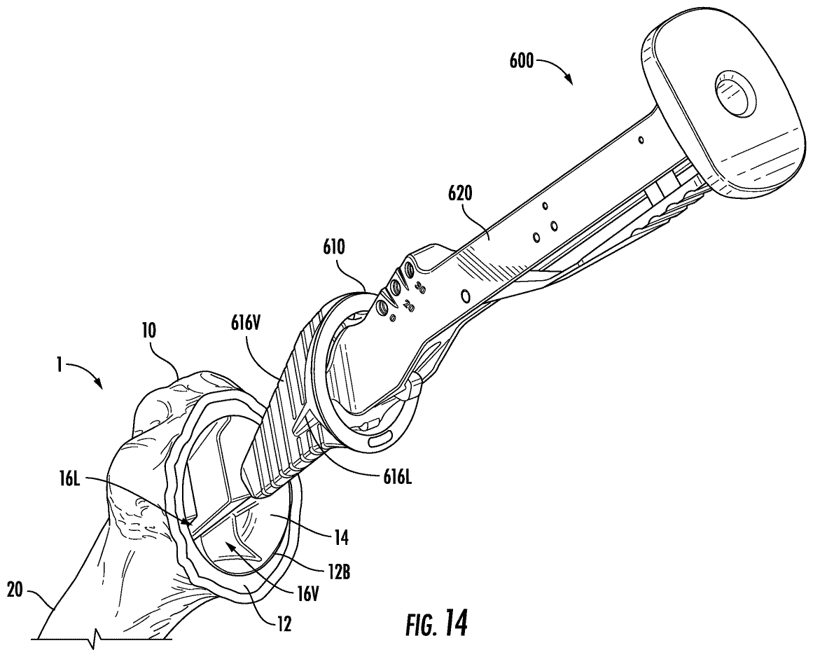

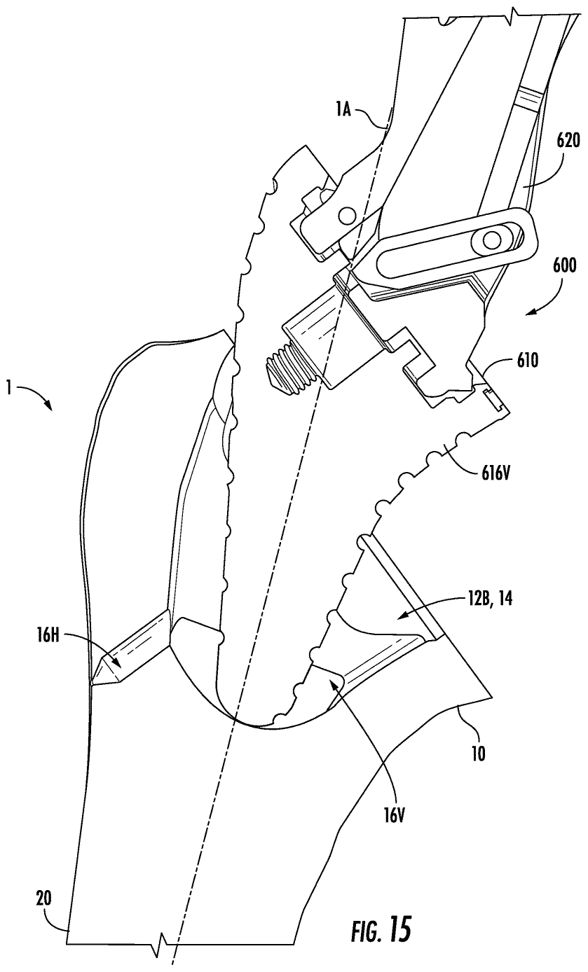

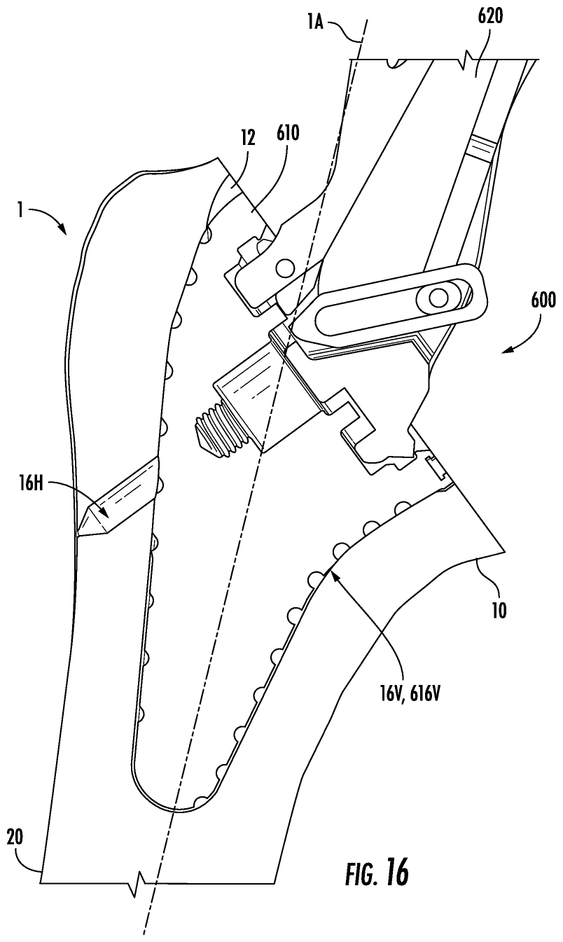

After the broaching procedure is complete, one or more compactors is/are applied to the broached implantation site 12B to form a partially or fully compacted implantation site. FIG. 11 shows an example of such a first stage compactor, generally designated 500. The first stage compactor 500 has a compactor head 510 that is rigidly attached to a compactor stem 520. The compactor head 510 has a vertical fin 516V that, when in the aligned orientation shown in FIGS. 12 and 13, is aligned with the vertical alignment slot 16V of the broached implantation site 12B shown in FIG. 10. The compactor head 510 also has a lateral fin 516L that extends away from the vertical fin 516V, on opposite sides of the vertical fin 516V. When the first stage compactor 500 is in the aligned orientation shown in FIGS. 12 and 13, the lateral fin 516L is aligned with the lateral alignment slot 16L of the broached implantation site 12B shown in FIG. 10. The vertical fin 516V and the lateral fin 516L intersect each other (e.g., at about 90°), such that the vertical fin 516V is bisected by the lateral fin 516L or, stated differently, the lateral fin 516L is bisected by the vertical fin 516V. The vertical fin 516V and the lateral fin 516L extend, when in the fully engaged position shown in FIG. 13, deeper than the vertical alignment slot 16V and the lateral alignment slot 16L, respectively. Thus, when the first stage compactor 400 is in the fully engaged position shown in FIG. 13, the vertical fin 516V will have enlarged the vertical alignment slot 16V to be larger than the vertical alignment slot 16V of the broached implantation site 12B, which was formed initially by the protruding ridge 316 of the broach 300, and the lateral fin 516L will have enlarged the lateral alignment slot 16L to be larger than the lateral alignment slot 16L of the broached implantation site 12B, which was initially formed by the insertion of the osteotome 400 into and at least partially through the osteotome slot 312 of the broach 300.

During each subsequent compaction procedure, the respective depths of the vertical alignment slot 16V and of the lateral alignment slot 16L are increased by insertion of compactors (e.g., 500, 600) having progressively larger vertical fins 516V, 616V and lateral fins 516L, 616L than the previously used compactor.

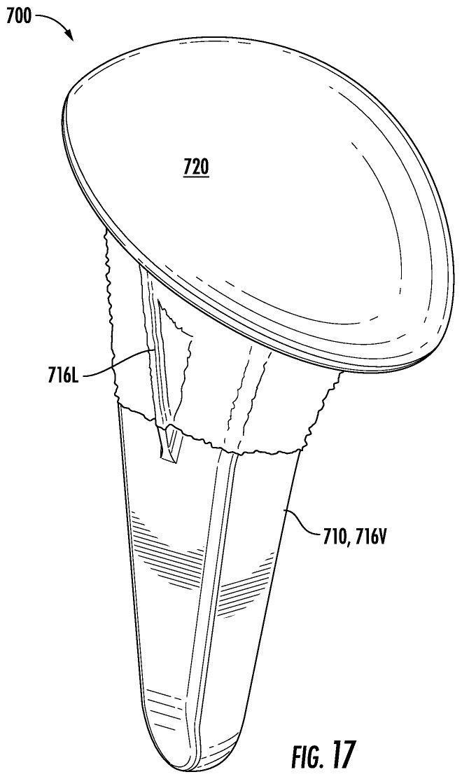

An example of a second stage compactor, generally designated 600, is shown in FIGS. 14-16. The second stage compactor 600 has a compactor head 610 that is rigidly attached to a compactor stem 620. The second stage compactor 600 has lateral and vertical fins 616L, 616V that are generally arranged at the same angle to each other as the lateral and vertical fins 516L, 516V of the first stage compactor 500, but the lateral and vertical fins 616L, 616V of the second stage compactor 600 have a more elongated shape than the lateral and vertical fins 516L, 516V of the first stage compactor 500, so as to progressively enlarge and elongate the lateral and vertical alignment slots 16L, 16V in the implantation cavity of the implantation site 12, this implantation cavity extending within and through the humeral head 10 and substantially coaxially towards and into the humeral shaft 20. This sequential enlarging of the lateral and vertical alignment slots 16L, 16V continues until a final compactor, which has lateral and vertical fins that are substantially the same size as (e.g., the same size as, or within 10% or 5% of the size thereof in any dimension) the corresponding alignment features of the prosthesis base that is to be implanted in the implantation site 12, is inserted into the implantation site 12, thus forming a fully compacted implantation site that is ready for insertion of the prosthesis 700 therein. In this non-limiting example, the second stage compactor 600 is the “final compactor” described herein. However, in some examples, additional compactors (e.g., third, fourth, etc.) may be provided to sequentially shape the implantation cavity during a series of sequentially performed compaction procedures or steps.

FIG. 17 shows an example humeral prosthesis, generally designated 700, with a prosthesis base 710 that is inserted within (e.g., implanted within) the implantation cavity at the implantation site. The prosthesis 700 has a prosthetic humeral head 720 that is attached (e.g., rigidly or permanently, or in a removable manner) to the prosthesis base 710. The prosthesis base 710 has lateral and vertical fins 716L, 716V that are formed to align with the lateral and vertical alignment slots 16L, 16V, respectively, that are formed within the implantation cavity of the fully compacted implantation site that is produced during the final compaction procedure. FIG. 18 shows features of the prosthesis base 710 of the example humeral prosthesis 700 shown in FIG. 17, with the prosthetic humeral head 720 not shown in FIG. 18 to better illustrate the features of the prosthesis base 710. The example prosthesis 700 has a prosthetic humeral head 720 that, when fully inserted within the implantation cavity, sits substantially flush with (e.g., coplanar to) the resection plane that defines the implantation site 12 on the outer surface of the humeral head 10, such that the prosthetic humeral head 720 acts as an anatomically correct replacement for the portion of the humeral head 10 that was resected.

The prosthesis base 710 has a stem, or vertical fin 716V, that extends through the humeral head 10 and, preferably, into the humeral shaft 20, at least in part, when the prosthesis base 710 is implanted within the implantation cavity. The vertical fin 716V is substantially the same size and shape as the vertical alignment slot 16V. The prosthesis base 710 has lateral fins 716L that extend from the vertical fin 716V in positions corresponding to the lateral alignment slots 16L. The prosthesis base 710 has a ridge area 18 that fits within a region of the vertical alignment slot 16V that is formed by the extended edge 316EE of the protruding ridge 316 of the broach 300 during the broaching procedure, or step. The prosthesis base 710 also has a bowl portion 714 that is positioned within the bowl region 14 of the base implantation site 12, such that the outermost surface of the prosthesis base 710 is substantially coplanar with the plane formed by the initial resection of the portion of the humeral head 10. The general shape of the bowl profile 714P of the bowl portion 714 is shown in broken lines in FIG. 18.

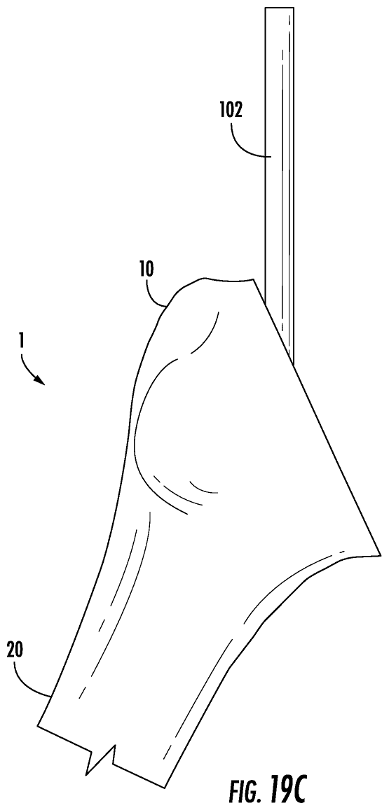

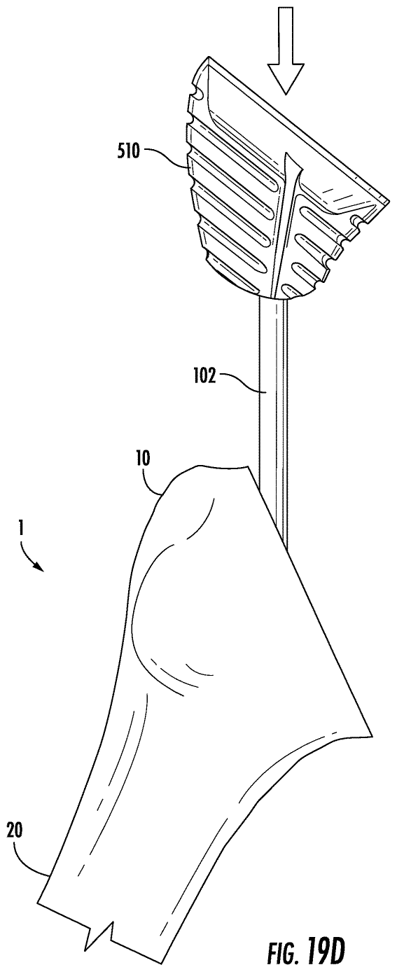

FIGS. 19A-19D show features of a further alternate example of a broach, generally designated 300, that can be used as part of an alternate method of performing the broaching procedure discussed elsewhere herein in relation to FIGS. 6-9. The broach 300 shown can be used together with or as an alternative to the broaches 300 shown in FIGS. 6-9. According to this example of FIGS. 19A-19D, the broach 300 is used, when in the position shown in FIG. 19B (corresponding to the position shown in FIG. 7), to guide insertion of a second guide pin 102 into the humeral head 10 and/or the humeral shaft 20. The second guide pin 102 is inserted through the broach 300 and into the implantation site 12 at an angle so as to be at substantially the same angle, relative to the longitudinal humeral axis 1A, as the lateral alignment slot(s) 16L that are shown in FIG. 10. The second guide pin 102 may, in some instances, be inserted substantially parallel to the longitudinal humeral axis 1A. In some instances, the second guide pin 102 may be inserted through the broach 300 without first removing the first guide pin 100 (e.g., the pin 100 shown installed in FIG. 2). It is preferred, however, if the first guide pin 100 is removed before the second guide pin 102 is installed, so that the second guide pin 102 can be installed through the center of the implantation site 12, so as to reduce off-axis effects.

The second guide pin 102 is installed to guide movement of the compactors (see 500, 600, FIGS. 11-16) relative to the implantation site 12, providing a mechanism for ensuring mechanical alignment of such compactors 500, 600 relative to the implantation site 12 during the subsequent compaction steps. This use of the second guide pin 102 to guide alignment of the compactors 500, 600 along the longitudinal humeral axis 1A is shown in FIG. 19D. For ease of illustration, only the first compactor head 510 is shown in FIG. 19D. Advantageously, to allow for removal of the first guide pin 100 from the implantation site 12 prior to the insertion of the second guide pin 102 into the implantation site 12, the broach stem 320 may be removably attached to the broach head 310, thereby allowing the broach head 310 to remain in place within the implantation site 12 while the broach stem 320 is disconnected from the broach head 310 and the first guide pin 100 is removed from the implantation site 12. This removable attachment of the broach stem 320 and broach head 310 can be achieved by a threaded interface (e.g., in the manner of a screw and nut). After the first guide pin 100 is removed, the second guide pin 102 is installed in the implantation site 12 through the broach head 310. The broach stem 320 may be reattached to the broach head 310 before installation of the second guide pin 102 in the implantation site 12. The broach head 310 and the broach stem 320 may, when attached together, thus form a continuous channel (e.g., bore 340, see FIGS. 7 and 8) that passes through the broach head 310 and broach stem 320 for guiding the second guide pin 102 into the implantation site, as shown in FIG. 19B. Once the second guide pin 102 has been inserted in the implantation site 12, the broach 302 is removed from the implantation site 12 in the direction defined by the longitudinal axis of the second guide pin 102. The second guide pin 102 can then be used to guide the movement of a compactor 500, 600 on/into the implantation site 12.

While the present disclosure refers to certain examples, numerous modifications, alterations, and changes to the described examples are possible without departing from the sphere and scope of the present disclosure. Accordingly, it is intended that the present disclosure not be limited to the described examples, but that it has the full scope defined by the language of the specification, and equivalents thereof, as would be understood by persons having ordinary skill in the art. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the descriptions of such examples herein are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one,” “one or more,” and “and/or,” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A broaching system configured for use in preparing an implantation site for implantation of a humeral prosthesis therein, the broaching system comprising at least:
a broach comprising:
a broach head;
a broach stem; and
an osteotome slot; and
an osteotome that is configured for removable insertion into and through the osteotome slot for forming one or more alignment features within the implantation site to aid a surgeon to properly align a compactor with the implantation site;
wherein the broach stem and the broach head each have a bore formed therein that is configured to receive therein a pin that is implanted within the implantation site, the bore being continuous and uninterrupted within and between the broach stem and the broach head.

2. The broaching system according to claim 1, wherein the osteotome slot is formed at an inclined, non-zero angle relative to a longitudinal axis of the broach stem.

3. The broaching system of claim 1, wherein the osteotome slot is continuous and uninterrupted from the broach stem to and through the broach head.

4. The broaching system of claim 1, wherein the osteotome has a length such that, when the osteotome is inserted fully into the osteotome slot, a distal portion of the osteotome protrudes out of the osteotome slot and into the implantation site to form the one or more alignment features.

5. (canceled)

6. The broaching system of claim 1, wherein the osteotome has a U-shaped profile, with two legs that extend substantially parallel to each other and a base that extends between and connects the two legs, such that the two legs are spaced apart from each other by the base.

7. The broaching system of claim 6, wherein the osteotome slot is formed around and is not coincident with the bore.

8. The broaching system of claim 1, wherein the broach head has a protruding ridge that extends away from the broach stem and is configured for forming a further alignment feature within the implantation site.

9. The broaching system of claim 8, wherein the protruding ridge has, on one side thereof, a curved edge and, on the opposite side thereof, an inclined edge that forms an obtuse angle relative to the bottom surface of the protruding ridge that connects the curved edge to the inclined edge.

10. The broaching system of claim 1, wherein the one or more alignment features extend substantially coaxially with a longitudinal axis of a humerus in which the implantation site is formed.

11. A method for performing a broaching procedure to prepare an implantation site for implantation of a humeral prosthesis therein, the method comprising:
providing a broach comprising:
a broach head including a bore formed therein, the bore being continuous and uninterrupted within the broach head;
a broach stem including a bore formed therein, the bore being continuous and uninterrupted within the broach stem and between the broach stem and the broach head; and
an osteotome slot;
providing an osteotome;
implanting a pin within the implantation site to maintain a prescribed alignment of the broach to the implantation site;
positioning the broach head and the broach stem onto the pin so that the pin is received within the bore of the broach head and the bore of the broach stem; and
inserting the osteotome into and through the osteotome slot for forming one or more alignment features within the implantation site to aid a surgeon to properly align a compactor with the implantation site;
wherein the osteotome is inserted within the osteotome slot in a manner than allows subsequent removal of the osteotome from the osteotome slot without damaging the one or more alignment features formed by the osteotome.

12. The method according to claim 11, wherein the osteotome slot is formed at an inclined, non-zero angle relative to a longitudinal axis of the broach stem.

13. The method of claim 11, wherein the osteotome slot is continuous and uninterrupted from the broach stem to and through the broach head.

14. The method of claim 11, wherein the osteotome has a length such that, when the osteotome is inserted fully into the osteotome slot, a distal portion of the osteotome protrudes out of the osteotome slot and into the implantation site to form the one or more alignment features.

15. (canceled)

16. The method of claim 11, wherein the osteotome has a U-shaped profile, with two legs that extend substantially parallel to each other and a base that extends between and connects the two legs, such that the two legs are spaced apart from each other by the base.

17. The method of claim 16, wherein the osteotome slot is formed around and is not coincident with the bore.

18. The method of claim 11, wherein the broach head has a protruding ridge that extends away from the broach stem for forming a further alignment feature within the implantation site.

19. The method of claim 18, wherein the protruding ridge has, on one side thereof, a curved edge and, on the opposite side thereof, an inclined edge that forms an obtuse angle relative to the bottom surface of the protruding ridge that connects the curved edge to the inclined edge.

20. The method of claim 11, wherein the one or more alignment features extend substantially coaxially with a longitudinal axis of a humerus in which the implantation site is formed.
